(19) United States
(12) Patent Application Publication
(10) Pub. No.: US 2026/0076827 A1
(43) Pub. Date: Mar. 19, 2026

(54) Adaptive Vacuum Booster for Phacoemulsification Operations

(71) Applicant: JOHNSON & JOHNSON SURGICAL VISION, INC., Irvine, CA (US)

(72) Inventors: Vadim Gliner, Haifa (IL); Assaf Govari, Haifa (IL)

(21) Appl. No.: 18/889,825

(22) Filed: Sep. 19, 2024

(51) Int. Cl. A61F 9/007; A61M 3/02

(52) U.S. Cl. CPC A61F 9/00745; A61M 3/0202; A61M 2210/0612

(57) ABSTRACT

A phacoemulsification system and a method for controlling a surgical system, the method including: providing an ophthalmic surgical system comprising: a phacoemulsification probe having a needle at a distal end and an ultrasonic transducer, wherein the needle is configured to be inserted into an eye of a patient; an aspiration line, wherein the aspiration line is fluidly coupled with the needle; an aspiration pump; a control device configured for a user to control the aspiration pump; and a processor; obtaining a value of intraocular pressure (IOP) within the eye; receiving a trigger from a user of the ophthalmic surgical system; and in response to receiving the trigger, operating the aspiration pump at a higher aspiration rate, for a period of time, wherein the period of time is determined in accordance with the value of the IOP.

### FIELD OF THE DISCLOSURE

This disclosure relates to phacoemulsification operations in general, and to boosting the vacuum intensity during an operation for a limited time while avoiding dangerous levels of intraocular pressure (IOP), in particular.

### BACKGROUND OF THE DISCLOSURE

A cataract is a clouding and hardening of the eye's natural lens, which often happens when people get older. A common treatment of a cataract is phacoemulsification cataract surgery. In the procedure, a clear cornea or scleral incision is made in the eye to access the internal structures and then a portion of the anterior surface of the lens capsule is removed to gain access to the cataract. The surgeon then uses a phacoemulsification probe, which is an ultrasonic handpiece with a needle and a coaxial sleeve at least partially surrounding the needle. The tip of the needle vibrates at ultrasonic frequency, which emulsifies the cataract lens. At a same time, a pump aspirates particles and fluid from the eye through the tip, wherein the aspirated fluids are replaced with irrigation of a balanced salt solution via the sleeve to maintain the intraocular pressure in the anterior chamber of the eye. After removing the cataract with phacoemulsification, the softer outer lens cortex is removed with suction. An intraocular lens (IOL) is then introduced into the empty lens capsule restoring the patient's vision.

### BRIEF DESCRIPTION OF THE DRAWINGS

The present disclosure will be more fully understood from the following detailed description of the examples thereof, taken together with the drawings, in which:

FIG. 1 is a partly pictorial, partly block diagram view of a phacoemulsification system constructed and operative in accordance with some examples of the disclosure;

FIGS. 2A-2D are pictorial views of a tip of a phacoemulsification probe inside a patient's eye and associated lens particles, in accordance with some examples of the disclosure;

FIG. 3A is a schematic illustration of side view of a progressive cavity pump (PCP), in accordance with some examples of the disclosure;

FIG. 3B is a graph of the Revolutions Per Minute (RPM) of a rotor of a PCP after a booster trigger has been identified, in accordance with some examples of the disclosure; and

FIG. 4 is a flowchart of steps in a method for an adaptive vacuum booster for phacoemulsification operations, in accordance with some examples of the disclosure; and

FIG. 5 is a schematic block diagram of an example of a computing platform for providing an adaptive vacuum booster for phacoemulsification operations, in accordance with some examples of the disclosure.

### DETAILED DESCRIPTION OF EXAMPLES

In the following description, numerous specific details are set forth in order to provide a thorough understanding of the present disclosure. It will be apparent to one skilled in the art, however, that the present disclosure may be practiced without these specific details. In other instances, well-known circuits, control logic, and the details of computer program instructions for conventional algorithms and processes have not been shown in detail in order not to obscure the present disclosure unnecessarily.

Software programming code, which embodies aspects of the present disclosure, is typically maintained in permanent storage, such as a computer readable medium. In a client-server environment, such software programming code may be stored on a client or a server. The software programming code may be embodied on any of a variety of known media for use with a data processing system. This includes, but is not limited to, magnetic and optical storage devices such as disk drives, magnetic tape, compact discs (CD's), digital video discs (DVD's), and computer instruction signals embodied in a transmission medium with or without a carrier wave upon which the signals are modulated. For example, the transmission medium may include a communications network, such as the Internet. In addition, while the disclosure may be embodied in computer software, the functions necessary to implement the disclosure may alternatively be embodied in part or in whole using hardware components such as application-specific integrated circuits or other hardware, or some combination of hardware components and software.

In the description below, the term “about” as related to numerical values may include values that are in the range of +/−10% of the indicated value.

### Overview

A cataract is removed by a phacoemulsification procedure, in which an ultrasonic handpiece having a needle is inserted into a patient's eye, wherein the tip of the needle can vibrate at ultrasonic frequency, to emulsify the cataract.

The vibrations cause particles of the lens to be released from the lens. The particles, together with fluids, are typically aspirated via an aspiration line running through or coupled with the needle via one or more pumps.

The one or more pumps have two major roles: first to draw the particle to the needle and attach it to the tip of the needle such that the particle can be broken into smaller particles via ultrasound, and second to aspire the smaller particles out of the eye.

The one or more pumps may be of one or more types.

A first type of pump is a vacuum based pump, e.g., a Venturi type pump, which maintains vacuum within a cell, such that when its valve opens to a space the pump is coupled with, the vacuum is instantly applied to the space, at full intensity. This behavior profile has both advantages and disadvantages: it is fast creating great followability, but at the same time it is less controllable, which may lead to damage to the eye. The damage may be caused by a vacuum surge occurring when a particle that occludes the tip of the needle causes vacuum to build up in the aspiration line behind the particle, such that when the particle breaks up, the buildup of vacuum causes a rush of fluid out of the eye and chamber collapse. However, such aggressive aspiration is sometimes needed, for example when the user such as a physician performing the operation, notices a particle that needs to be aspirated or brought to the distal end of the needle to be emulsified.

A second type of pump is a flow based pump, e.g., a peristaltic pump. The advantage of the flow based pump is its controllability and holdability. Its disadvantage is that vacuum is only created when a particle occludes or partially occludes the distal end of the needle, and thus the vacuum may not be created fast enough when the need for an aspiration boost arises.

Another example of a flow based pump is a progressive cavity pump (PCP), which comprises a rotor and a stator. By rotating the rotor, the PCP can build vacuum, wherein the vacuum intensity depends on the rotation speed.

Thus, in some examples, a phacoemulsification system may be equipped with a progressive cavity pump for controllability and holdability, but there is a need for a boost in vacuum similar to a vacuum based pump.

The instant vacuum (or vacuum boost) may be built and applied in certain circumstances, for example upon an explicit trigger provided by a user. The trigger may be, for example, pressing a pedal or button, pressing a pedal with at least a predetermined force, or the like. In other examples, the trigger may be yawing a dual linear foot pedal to the left or right, or pressing a button on the foot pedal. In further examples, the trigger may be vocal and may take effect when a trigger word is identified within captured audio. In other examples, the vacuum may be applied in cases when the IOP increases sharply or to dangerous degrees, and normal control of the aspiration pump is insufficient to maintain the IOP in the desired range.

It is appreciated that sudden application of vacuum causes a decrease in the IOP, and that the longer the period of time the vacuum is applied, the sharper the IOP decreases. Thus, in order to maintain the IOP within a desired range, and particularly to make sure it is above a minimal acceptable value, the period of time for which the vacuum is applied may be determined in accordance with the IOP in the eye as measured immediately before the before the vacuum boost is activated, for example at most a predetermined period of time before. For example, it may be required to maintain an IOP between 24 mmHg and 90 mmHg. Thus, if the IOP is about 40 mmHg immediately before the vacuum boost is applied, the vacuum boost may be applied for about 0.5 seconds, such that the IOP does not drop below 24 mmHg. However, if the IOP immediately before the vacuum boost is applied is about 90 mmHg, the vacuum boost may be applied for about 5 seconds without risking a decrease in the IOP to below 24 mmHg.

Thus, upon receiving a trigger, a higher vacuum (e.g., a maximum achievable vacuum of the pump as determined by the maximal rotation speed of the rotor or maximum vacuum setting), may be applied for a period of time determined in accordance with the IOP measured immediately before the vacuum boost application is initiated.

It is appreciated that further criteria may be defined for identifying situations in which it is required to stop the aspiration in order to avoid the vacuum surge.

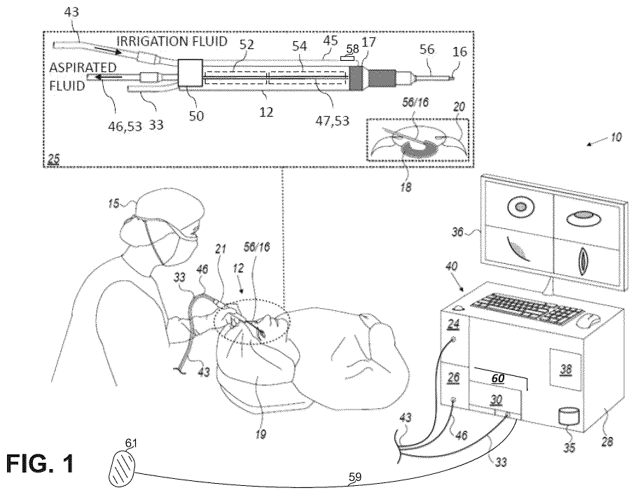

Reference is now made to FIG. 1 that is a partly pictorial, partly block diagram view of a phacoemulsification system 10 constructed and operative in accordance with some examples of the present disclosure.

Phacoemulsification system 10 comprises a phacoemulsification probe 12 (e.g., a handpiece). In some examples, phacoemulsification probe 12 may be replaced by any suitable medical tool. As seen in the pictorial view of system 10, and in inset 25, phacoemulsification probe 12 comprises a needle 16, a probe body 17, and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, irrigation sleeve 56 may have one or more side ports at, or near, the distal end to allow irrigation fluid to flow towards the distal end of the phacoemulsification probe 12 through the fluid pathway and out of the port(s). Needle 16 of phacoemulsification probe 12 is configured for insertion into a lens capsule 18 of an eye 20 of a patient 19 by a physician 15, to remove a cataract. While needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA.

In the example of FIG. 1, during the phacoemulsification procedure, eye fluid and waste matter (e.g., emulsified parts of the cataract) are aspirated via an aspiration channel 47, which extends from the hollow of needle 16 through phacoemulsification probe 12, and then via an aspiration tubing line 46 to a collection receptacle (not shown) in console 28. The aspiration is affected by a pumping subsystem 24, also comprised in console 28. Aspiration tubing line 46 and aspiration channel 47 are collectively referred to as an aspiration line 53. Aspiration line 53 is therefore coupled with needle 16 and pumping subsystem 24, which is configured to remove the fluid and waste matter from eye 20 via aspiration line 53.

Also, during the phacoemulsification procedure, a pumping subsystem 26 comprised in a console 28 pumps irrigation fluid from an irrigation reservoir (not shown) to irrigation sleeve 56 to irrigate eye 20. The irrigation fluid is pumped via an irrigation tubing line 43 running from console 28 to an irrigation channel 45 of probe 12, the distal end of irrigation channel 45 including the fluid pathway in irrigation sleeve 56. In another example, pumping subsystem 26 may be coupled or replaced with a gravity fed irrigation source such as a balanced salt solution bottle/bag.

In some examples, pumping subsystem 24 and pumping subsystem 26 may be combined into a unified fluidics system.

System 10 may include a chamber stabilization system (CSS) 50 (which in an example, may be removable), which may include one or more valves to regulate the flow of fluid in irrigation tubing line 43, irrigation channel 45, and/or aspiration line 53 as well as one or more sensors. Parts of irrigation channel 45 and aspiration line 53 are disposed in probe body 17 and parts are disposed in CSS 50. CSS 50 may also be external to the handpiece but fluidly coupled with aspiration line 53, irrigation channel 45, and irrigation line 43, e.g., proximal to the proximal end of handpiece 12. Phacoemulsification probe 12 may include other elements, such as an ultrasonic actuator 52, e.g., piezoelectric crystal(s), coupled with a horn 54 configured to support needle 16 and drive vibration of needle 16 to emulsify the lens of eye 20. Ultrasonic actuator 52 is configured to vibrate needle 16 in a resonant vibration mode. The vibration of needle 16 is used to break a cataract into small pieces during a phacoemulsification procedure.

Console 28 may comprise an ultrasonic (e.g., piezoelectric) drive module 30, coupled with ultrasonic actuator 52, using electrical wiring running in a cable 33. Drive module 30 is controlled by a controller 38 and conveys processor-controlled driving signals via cable 33 to, for example, maintain needle 16 at predetermined vibration amplitude. Drive module 30 may be realized in hardware or software, for example, in a proportional-integral-derivative (PID) control architecture. Controller 38 may also be configured to receive signals from sensors in phacoemulsification probe 12 and/or in CSS 50 and control one or more valves to regulate the flow of fluid in irrigation channel 45 and/or aspiration line 53. In some examples, at least some of the functionality of controller 38 may be implemented using a controller disposed in phacoemulsification probe 12 (e.g., in CSS 50).

Controller 38 may receive user-based commands via a user interface 40, which may include setting a vibration mode and/or frequency of ultrasonic actuator 52, and setting or adjusting an irrigation and/or aspiration rate of pumping subsystems 24/26. In some examples, user interface 40 and a display 36 may be combined as a single touch screen displaying a graphical user interface.

In some examples, the user interface may allow physician 15 to apply higher aspiration, for example maximal aspiration of the pump for a limited period of time, for example when physician 15 notices one or more particles that need to be broken and aspired. For example, physician 15 may use foot pedal 61 as a means of control. Foot pedal 61 may be connected by cable 59 to (or be wirelessly coupled with) controller 38 which, upon receiving an indication of physician 15 pressing the pedal, would activate pumping subsystem 24 at a different speed, e.g., its full speed, for a limited period of time. Additionally, or alternatively, controller 38 may receive the user-based commands from controls located on handle 21 of probe 12. For example, pressing on button 58 may initiate the activation of pumping subsystem 24 at a different speed. In some examples, button 58 may be located anywhere along probe 12, including farther from the usual position of any of the fingers of physician 15, to avoid unintentional pressing.

In further examples, a vocal trigger may be received, where a microphone (not shown) is provided and adapted to capture audio from physician 15. A processor associated with controller 38 may receive and analyze the audio and activate pumping subsystem 24 at a different speed upon recognizing a trigger word or phrase said by physician 15, such as “max please”, or the like.

Some or all of the functions of controller 38 may be combined in a single physical component or, alternatively, implemented using multiple physical components. These physical components may comprise hard-wired or programmable devices, or a combination of the two. In some examples, at least some of the functions of controller 38 may be carried out by suitable software stored in a memory device 35. This software may be downloaded to a device in electronic form, over a network, for example. Alternatively, or additionally, the software may be stored in tangible, non-transitory computer-readable storage media, such as optical, magnetic, or electronic memory.

The system shown in FIG. 1 may include further elements which are omitted for clarity of presentation. For example, physician 15 typically performs the procedure using a stereomicroscope. Physician 15 may use other surgical tools in addition to probe 12, which are also not shown, in order to maintain clarity and simplicity of presentation.

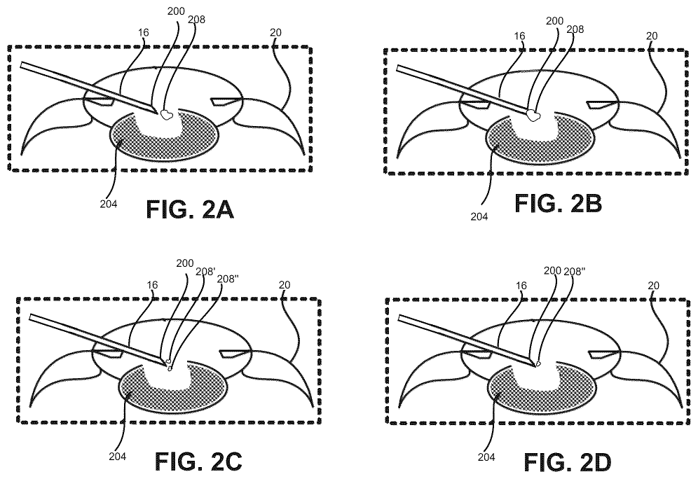

Reference is now made to FIG. 2A-2D, showing an exemplary scenario of breaking and aspiring a particle, in accordance with examples of the disclosure.

FIG. 2A shows an illustration of needle 16 inside eye 20 of patient 19. FIG. 2A further shows tip 200 and particle 208 released from lens 204 during the operation. Particle 208 is too large to be aspired by needle 200, thus it first needs to be broken into smaller particles.

As shown in FIG. 2B, due to the aspiration applied through needle 200, particle 208 is drawn to the tip of needle 200, where it can be broken into smaller particles.

FIG. 2C shows particle 208 as broken into first particle 208′ and second particle 208″. It is appreciated that one breaking may not be sufficient to break the particle into sub-particles which are all of a size that can be aspired. Therefore, further breaking iterations may be required.

FIG. 2D shows the situation where due to the applied vacuum, particle 208′ has been aspired into needle 200, and particle 208″ got closer to the tip of needle 200 and is about to be aspired, too.

When the physician notices one or more particles that need to be aspired, the physician may activate the aspiration pump at a different speed. Then the particles, if of a suitable size, can be aspired into the needle. If the particles are too large, the aspiration may bring them to the tip of the needle, where they can be further broken and aspired. The pump may be activated at a different speed for a period of time determined in accordance with the IOP within the eye as measured prior to the activation, such that the activation will not cause the IOP to drop below a predetermined threshold.

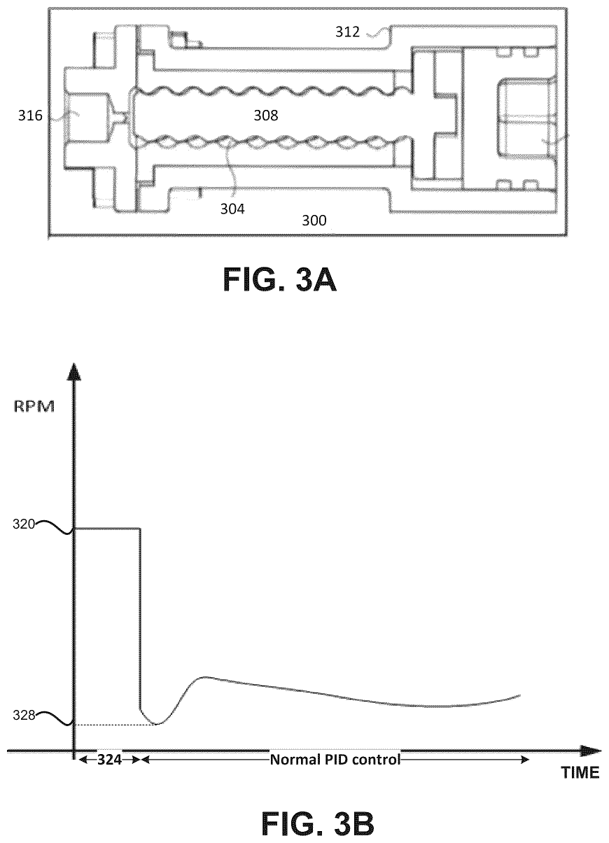

Referring now to FIG. 3A, showing a schematic illustration of side view of a PCP, in accordance with some examples of the disclosure.

The PCP may be embedded in cartridge 300 which may be coupled with pumping subsystem 24 or a fluidics system used for irrigation as well as aspiration.

The PCP may comprise one or two stators 304 including an aspiration stator and optionally an irrigator stator in fluidic communication with respective one or more ports 316, coupled with a collection container and optionally with an irrigation reservoir and with corresponding probes.

Cartridge 300 further comprises one or two rotors 308 rotatably disposed, respectively, within stators 304, wherein the rotors may be metallic, polymeric, or any other suitable material.

The rotation of rotor 308 within stators 304 causes fluid and debris to flow from the eye, through the cartridge and the respective port to the collection container.

It is appreciated that increasing or decreasing the rotation speed at which rotor 308 rotates within stator 304 increases or decreases, respectively, the speed at which fluid and debris from the eye are aspired. It is also appreciated that rotating rotor 308 at the highest possible speed instantaneously rather than gradually, will cause instantaneous maximal aspiration of the fluid and debris from the eye.

Referring now to FIG. 3B, showing a graph of the Revolutions Per Minute (RPM) of rotor 308 after a trigger has been identified, in accordance with some examples of the disclosure.

In response to receiving the trigger, the RPM is instantly increased to the higher (e.g., maximal possible) value 320, to provide a vacuum boost. Width 324 of the boost period is determined in accordance with the latest value of the IOP measured prior to operating the higher RPM. Thus, a higher IOP provides for operating the boost for a longer period of time, since the pressure drop will not lead to a vacuum surge, and vice versa. For example, if the IOP is about 90 mmHg, the boost period can be about 5 seconds, while if the IOP is about 40 mmHG, the boost period can be about 0.5 seconds.

Once the predetermined boost period is over, PID control may be resumed. First the RPM may be significantly reduced, for example to a value 328 which may be between about 10% and about 30% of the higher value 320, to enable restoration of the IOP. Then normal PID control may be resumed, aimed at maintaining the IOP at a predetermined value or range, for example between 60 mmHG and 90 mmHG.

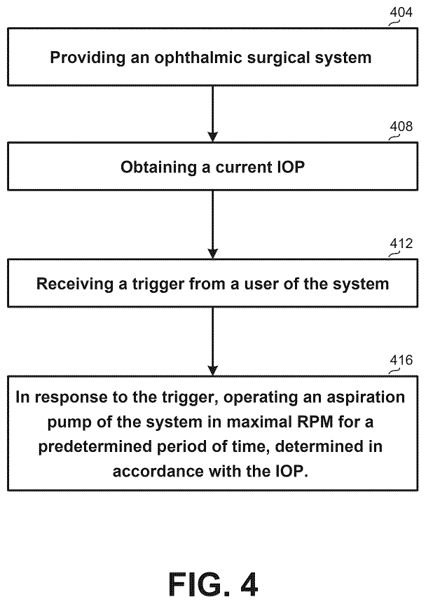

Referring now to FIG. 4, showing a flowchart of steps in a method for an adaptive vacuum booster for phacoemulsification operations, in accordance with some examples of the disclosure.

At step 404, an ophthalmic surgical system, for example as described above, may be provided to a customer, such as a hospital or another institute, to be operated by a user such as a physician.

At step 408, the IOP within the eye may be obtained, for example continuously or at predetermined time intervals during the phacoemulsification operation, and provided to the processor.

At step 412, a trigger may be received from the user of the system, for example using the I/O device. The trigger may indicate that the user wishes to boost the aspiration, for example to a maximal level, for a limited period of time.

The trigger may be received by the user pressing a pedal associated with the system. In some examples, the press may be required to be at a minimal predetermined force and or speed.

In further examples, the trigger may be received from the user pushing a button or control located on the probe, by capturing audio and analyzing the audio to identify a trigger word or phrase, or the like.

At step 416, upon receiving the trigger, the aspiration pump may be steered at a higher RPM, for example at its maximal RPM value, for a predetermined period of time. The period of time may be determined in accordance with the latest available IOP value as received at step 408. The period of time corresponding to each IOP value may be calculated by a function, obtained from a look up table, or the like. The period of time may also be shortened, and the boost may be stopped if the IOP measure drops below a predetermined value.

In an example, once the period of time is over, the RPM may be reduced to a set point, such as 10%-30% of the maximal RPM for a second predetermined period of time or until the IOP increases to a required value. In another example, the RPM may be set to a same value as before the vacuum boost was applied, for a second predetermined period of time or until the IOP increases to a required value. Following the second predetermined period of time, the RPM may be controlled to maintain the IOP at a predetermined value or range.

The method of FIG. 4 may be coded as a computer program product. The computer program product may include a computer readable storage medium (or media) having computer readable program instructions thereon for causing a processor to carry out aspects of the present invention.

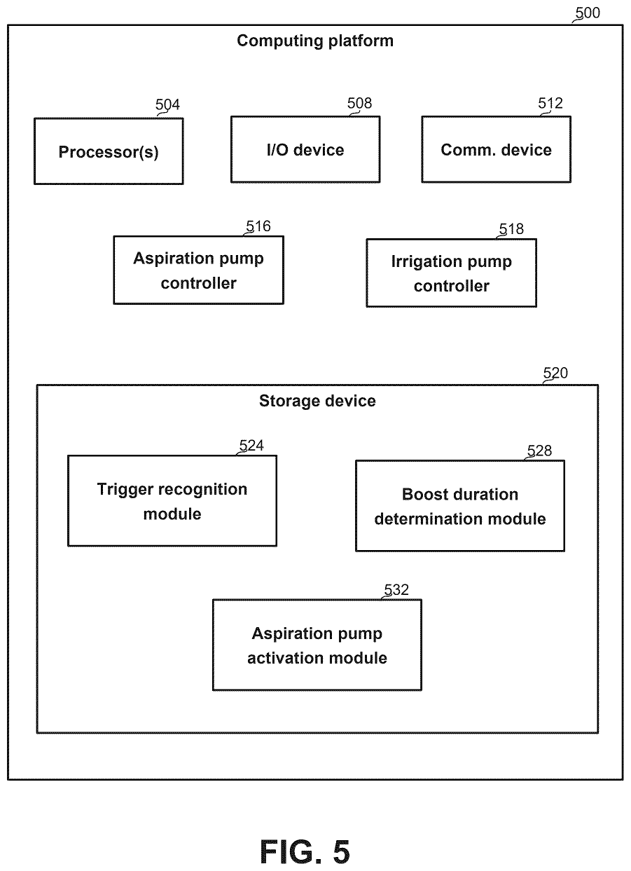

Referring now to FIG. 5, showing an example block diagram of a computing platform 500 for providing an adaptive vacuum booster for phacoemulsification operations, in accordance with some examples of the disclosure.

It is appreciated that computing platform 500 may be embedded within console 28, but may also be a standalone computing platform, or embedded elsewhere and be in operative communication with console 28.

Computing platform 500 may be implemented as one or more computing platforms which may be operatively connected to each other. For example, one or more remote computing platforms, which may be implemented for example on a cloud computer. Other computing platforms may be a part of a computer network of the associated organization. In other examples, all the functionality may be provided by one or more computing platforms all being a part of the organization network.

Computing platform 500 may comprise one or more processors 504, whether co-located on the same computing platform or not, and may be one or more Central Processing Units (CPU), microprocessors, electronic circuits, Integrated Circuits (IC) or the like. Processor 504 may be configured to provide the required functionality, for example by loading to memory and activating the software modules stored on storage device 520 detailed below.

Computing platform 500 may comprise an I/O device 508, for the user to define various parameters, e.g., minimal acceptable IOP, normal IOP for the operation, or the like. I/O device may include a display, a keyboard, a touchscreen, a mouse or another pointing device, or the like. I/O device 508 may also include a device for the user to initiate boosting of the PCP, such as a foot pedal, a control over the probe, a microphone and corresponding audio analysis device, or the like.

Computing platform 500 may comprise a communication device 512 for communicating with other devices or other computing platforms, for example obtaining IOP measurements, receiving a table of a function for calculating the boost period, boost amount, or the like. Communication module 512 may be adapted to interface with any communication channel such as Local Area Network (LAN), Wide Area Network (WAN), cellular network or the like, and use any relevant communication protocol.

Computing platform 500 may comprise an aspiration pump controller 516 for activating the aspiration pump at a required RPM, according to instructions received from processor 504.

Computing platform 500 may comprise an irrigation pump controller 518 for activating the irrigation pump as required to maintain the IOP in accordance with the aspiration pump controller, according to instructions received from processor 504.

Computing platform 500 may comprise a storage device 520, such as a hard disk drive, a Flash disk, a Random Access Memory (RAM), a memory chip, or the like. In some examples, storage device 520 may retain program code operative to cause processor 504 to perform acts associated with any of the modules listed below, or steps of the methods of FIG. 4 above. The program code may comprise one or more executable units, such as functions, libraries, standalone programs or the like, adapted to execute instructions as detailed below.

Alternatively, or additionally, the provided instructions may be stored on non-transitory tangible computer-readable media, such as magnetic, optical, or electronic memory.

Storage device 520 may comprise trigger recognition module 524, for receiving a trigger from pedal 61, control 58, or a vocal trigger obtained for example from an audio analysis module (not shown) processing audio captured by a microphone.

Storage device 520 may comprise boost duration determination module 528 for determining the period of time in which the aspiration pump needs to be operated at higher RPM value. The period of time may be determined in accordance with the latest available IOP measurement, and according to a look-up table, a function, or the like.

Storage device 520 may comprise aspiration pump activation module 532 for sending a command to aspiration pump controller 516 to activate the pump at the required RPM value at each point in time.

It is appreciated that the steps and modules disclosed above are in addition to the software, hardware, firmware, or other modules required for operating the probe, irrigating the eye, displaying the phacoemulsification process, performing other calculations required for example for operating the aspiration and irrigation systems, or the like.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembly instructions, instruction-set-architecture instructions, machine instructions, machine dependent instructions, microcode, firmware instructions, state-setting data, or either source code or object code written in any combination of one or more programming languages, programming languages such as Java, C, C++, Python, or others. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### EXAMPLES

### Example 1

A method for controlling a surgical system, comprising: providing an ophthalmic surgical system comprising: a phacoemulsification probe having a needle at a distal end and an ultrasonic transducer, wherein the needle is configured to be inserted into an eye of a patient; an aspiration line, wherein the aspiration line is fluidly coupled with the needle; an aspiration pump; a control device configured for a user to control the aspiration pump; and a processor; obtaining a value of intraocular pressure (IOP) within the eye; receiving a trigger from a user of the ophthalmic surgical system; and in response to receiving the trigger, operating the aspiration pump at a higher aspiration rate, for a period of time, wherein the period of time is determined in accordance with the value of the IOP.

### Example 2

The method according to example 1, wherein the aspiration pump is a Progressive Cavity Pump (PCP).

### Example 3

The method according to any of examples 1-2, wherein the higher aspiration rate is a maximal aspiration rate of the aspiration pump.

### Example 4

The method according to any of examples 1-3, wherein the value of the IOP used for determining the period of time is taken at a predetermined time prior to operating the aspiration pump at the higher aspiration rate.

### Example 5

The method according to any of examples 1-4, wherein the trigger comprises a press on a pedal of the system, the press exceeding a predetermined speed or force.

### Example 6

The method according to any of examples 1-5, wherein the trigger comprises yawing a dual linear foot pedal to the left or right, or pressing a button on a foot pedal.

### Example 7

The method according to any of examples 1-6, wherein the trigger is a vocal trigger.

### Example 8

The method according to any of examples 1-7, wherein the limited period of time is determined to limit the IOP to a minimal acceptable value.

### Example 9

The method according to any of examples 1-8, wherein the limited period of time is longer for a higher value of IOP, and shorter for a lower value of IOP.

### Example 10

The method according to any of examples 1-9, wherein for IOP of about 90 mmHg the limited period of time is about 5 seconds.

### Example 11

The method according to any of examples 1-10, wherein for IOP of about 40 mmHg the limited period of time is about 0.5 seconds.

### Example 12

A phacoemulsification system, comprising: a phacoemulsification probe having a needle at a distal end, and an ultrasonic transducer, wherein the needle is configured to be inserted into an eye of a patient; an aspiration line, wherein the aspiration line is fluidly coupled with the needle; an aspiration pump; a control device configured for a user to control the aspiration pump; and a processor, configured to: obtaining a value of intraocular pressure (IOP) within the eye; receiving a trigger from a user of the ophthalmic surgical system; and in response to receiving the trigger, operating the aspiration pump at a higher aspiration rate, for a period of time, wherein the period of time is determined in accordance with the value of the IOP.

### Example 13

The phacoemulsification system according to example 12, wherein the aspiration pump is a Progressive Cavity Pump (PCP).

### Example 14

The phacoemulsification system according to any of examples 12-13, wherein the higher aspiration rate is a maximal aspiration rate of the aspiration pump.

### Example 15

The phacoemulsification system according to any of examples 12-14, wherein the trigger comprises at least one item selected form the group consisting of: a press on a pedal of the system, the press exceeding a predetermined speed or force; yawing a dual linear foot pedal to the left or right; a press of a button on a foot pedal; and a vocal trigger.

### Example 16

The phacoemulsification system according to any of examples 12-15, wherein the limited period of time is determined to limit the IOP to a minimal acceptable value.

### Example 17

The phacoemulsification system according to any of examples 12-16, wherein the limited period of time is longer for a higher value of IOP, and shorter for a lower value of IOP.

### Example 18

The phacoemulsification system according to any of examples 12-17, wherein for IOP of about 90 mmHg the limited period of time is about 5 seconds.

### Example 19

The phacoemulsification system according to any of examples 12-18, wherein for IOP of about 40 mmHg the limited period of time is about 0.5 seconds.

### Example 20

A computer program product comprising a non-transitory computer readable storage medium retaining program instructions configured to cause a processor to perform actions, which program instructions implement: providing an ophthalmic surgical system comprising: a phacoemulsification probe having a needle at a distal end and an ultrasonic transducer, wherein the needle is configured to be inserted into an eye of a patient; an aspiration line, wherein the aspiration line is fluidly coupled with the needle; an aspiration pump; a control device configured for a user to control the aspiration pump; and a processor; obtaining a value of intraocular pressure (IOP) within the eye; receiving a trigger from a user of the ophthalmic surgical system; and in response to receiving the trigger, operating the aspiration pump at a higher aspiration rate, for a period of time, wherein the period of time is determined in accordance with the value of the IOP.

Although the examples described herein mainly address cardiac diagnostic applications, the methods and systems described herein can also be used in other medical applications.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A method for controlling a surgical system, comprising:
providing an ophthalmic surgical system comprising:
a phacoemulsification probe having a needle at a distal end and an ultrasonic transducer, wherein the needle is configured to be inserted into an eye of a patient;
an aspiration line, wherein the aspiration line is fluidly coupled with the needle;
an aspiration pump;
a control device configured for a user to control the aspiration pump; and
a processor;
obtaining a value of intraocular pressure (IOP) within the eye;
receiving a trigger from a user of the ophthalmic surgical system; and
in response to receiving the trigger, operating the aspiration pump at a higher aspiration rate, for a period of time,
wherein the period of time is determined in accordance with the value of the IOP.

2. The method of claim 1, wherein the aspiration pump is a Progressive Cavity Pump (PCP).

3. The method of claim 1, wherein the higher aspiration rate is a maximal aspiration rate of the aspiration pump.

4. The method of claim 1, wherein the value of the IOP used for determining the period of time is taken at a predetermined time prior to operating the aspiration pump at the higher aspiration rate.

5. The method of claim 1, wherein the trigger comprises a press on a pedal of the system, the press exceeding a predetermined speed or force.

6. The method of claim 1, wherein the trigger comprises yawing a dual linear foot pedal to the left or right, or pressing a button on a foot pedal.

7. The method of claim 1, wherein the trigger is a vocal trigger.

8. The method of claim 1, wherein the limited period of time is determined to limit the IOP to a minimal acceptable value.

9. The method of claim 1, wherein the limited period of time is longer for a higher value of IOP, and shorter for a lower value of IOP.

10. The method of claim 1, wherein for IOP of about 90 mmHg the limited period of time is about 5 seconds.

11. The method of claim 1, wherein for IOP of about 40 mmHg the limited period of time is about 0.5 seconds.

12. A phacoemulsification system, comprising:
a phacoemulsification probe having a needle at a distal end, and an ultrasonic transducer, wherein the needle is configured to be inserted into an eye of a patient;
an aspiration line, wherein the aspiration line is fluidly coupled with the needle;
an aspiration pump;
a control device configured for a user to control the aspiration pump; and
a processor, configured to:
obtaining a value of intraocular pressure (IOP) within the eye;
receiving a trigger from a user of the ophthalmic surgical system; and
in response to receiving the trigger, operating the aspiration pump at a higher aspiration rate, for a period of time,
wherein the period of time is determined in accordance with the value of the IOP.

13. The phacoemulsification system of claim 12, wherein the aspiration pump is a PCP.

14. The phacoemulsification system of claim 12, wherein the higher aspiration rate is a maximal aspiration rate of the aspiration pump.

15. The phacoemulsification system of claim 12, wherein the trigger comprises at least one item selected form the group consisting of: a press on a pedal of the system, the press exceeding a predetermined speed or force; yawing a dual linear foot pedal to the left or right; a press of a button on a foot pedal; and a vocal trigger.

16. The phacoemulsification system of claim 12, wherein the limited period of time is determined to limit the IOP to a minimal acceptable value.

17. The phacoemulsification system of claim 12, wherein the limited period of time is longer for a higher value of IOP, and shorter for a lower value of IOP.

18. The phacoemulsification system of claim 12, wherein for IOP of about 90 mmHg the limited period of time is about 5 seconds.

19. The phacoemulsification system of claim 12, wherein for IOP of about 40 mmHg the limited period of time is about 0.5 seconds.

20. A computer program product comprising a non-transitory computer readable storage medium retaining program instructions configured to cause a processor to perform actions, which program instructions implement:
providing an ophthalmic surgical system comprising:
a phacoemulsification probe having a needle at a distal end and an ultrasonic transducer, wherein the needle is configured to be inserted into an eye of a patient;
an aspiration line, wherein the aspiration line is fluidly coupled with the needle;
an aspiration pump;
a control device configured for a user to control the aspiration pump; and
a processor;
obtaining a value of intraocular pressure (IOP) within the eye;
receiving a trigger from a user of the ophthalmic surgical system; and
in response to receiving the trigger, operating the aspiration pump at a higher aspiration rate, for a period of time,
wherein the period of time is determined in accordance with the value of the IOP.
